(19) United States
(12) Patent Application Publication
(10) Pub. No.: US 2011/0065723 A1
(43) Pub. Date: Mar. 17, 2011

(54) COMPOSITIONS OF N-BENZYL-3-(4-CHLOROPHENYL)-2-[METHYL-[2-OXO-2-(3,4,5-TRIMETHOXYPHENYL)ACETYL]AMINO]-N-[3-(4-PYRIDYL)-1-[2-(4-PYRIDYL)ETHYL]PROPYL]PROPANAMIDE AND USES THEREOF

(75) Inventors: Trudy H. Grossman, Lexington, MA (US); Christopher Locher, Lexington, MA (US)

(73) Assignee: VERTEX PHARMACEUTICALS INCORPORATED, Cambridge, MA (US)

(21) Appl. No.: 12/879,269

(22) Filed: Sep. 10, 2010

Related U.S. Application Data
(60) Provisional application No. 61/241,435, filed on Sep. 11, 2009.

(51) Int. Cl. A61K 31/496; A61K 31/395; A61K 31/438; A61P 31/06; C12N 1/20

(52) U.S. Cl. 514/254.11; 514/183; 514/278; 435/253.1

(57) ABSTRACT

The present invention relates to compositions of N-benzyl-3-(4-chlorophenyl)-2-[methyl-[2-oxo-2-(3,4,5-trimethoxyphenyl)acetyl]amino]-N-[3-(4-pyridyl)-1-[2-(4-pyridyl)ethyl]propyl]propanamide (Timcodar) useful for the treatment of patients with mycobacterium infections such as Mycobacterium tuberculosis. The invention also provides methods of treating patients with tuberculosis.

### CROSS REFERENCE TO RELATED APPLICATIONS

This application claims the benefit of priority under 35 U.S.C. §119 to U.S. provisional patent application Ser. No. 61/241,435, filed Sep. 11, 2009, the entire contents of which are incorporated herein by reference.

### TECHNICAL FIELD OF THE INVENTION

The invention relates to compositions of N-benzyl-3-(4-chlorophenyl)-2-[methyl-[2-oxo-2-(3,4,5-trimethoxyphenyl)acetyl]amino]-N-[3-(4-pyridyl)-1-[2-(4-pyridyl)ethyl]propyl]propanamide (Timcodar) useful for the treatment of subjects with mycobacterium infections such as tuberculosis. The invention also provides methods of treating subjects with tuberculosis.

### BACKGROUND OF THE INVENTION

The infectious disease, tuberculosis (TB), is the leading cause of death worldwide from a single human pathogen, claiming more adult lives than diseases such as acquired immunodeficiency syndrome (AIDS), malaria, diarrhea, leprosy and all other tropical diseases combined (Zumla A, Grange J. B M J (1998) 316, 1962-1964). About one third of the world's population is currently infected with Mycobacterium tuberculosis (Mtb), the disease-causing agent; 10% of those infected will develop clinical diseases, particularly those who also have the human immunodeficiency virus (HIV) infection (Zumla A, Grange J. B M J (1998) 316, 1962-1964). Although the rate at which people are developing TB has declined, the number of cases continues to increase slowly, according to WHO figures. Hardest hit areas are in the developing world, where poverty, other diseases, and inadequate health care are factors. Killing about 1.6 million people annually, TB is the second leading infectious cause of death worldwide, after HIV/AIDS, and the leading cause in people infected with HIV. An estimated one-third of 40 million people with HIV are infected with TB.

HIV-infected people have a high risk for developing TB. But most people, if not HIV-infected, don't get TB when exposed to it. Of those who do, just a few percent develop active disease, while about 90% develop asymptomatic, noncontagious latent TB infections. Carried by nearly 2 billion people worldwide, latent TB infections can reactivate decades later, for instance when the immune system is suppressed. TB often manifests as pulmonary disease, but disseminated forms can affect almost all the body's organs.

The existing vaccine helps protect young children from developing serious disseminated forms of TB, but it is unreliable in preventing pulmonary TB in adolescents and adults. TB is curable when diagnosed adequately and treated appropriately, but this remains difficult. WHO recommends a treatment regimen for active, drug-susceptible TB consisting of four antibiotics—isoniazid, rifampicin (also called rifampin), ethambutol, and pyrazinamide—taken for two months, followed by isoniazid and rifampicin for another four. Latent TB infections often are treated with isoniazid alone for nine months.

Using multiple drugs with different modes of action prevents Mtb from developing resistance to any one drug. The extent of treatment is designed to purge a resilient and persistent bacterium adept at hiding from the immune system and lying dormant in the body for decades. Current HIV and TB therapies can be incompatible and thus are given separately or under strict monitoring.

In the most basic cases, patient compliance with the complicated, lengthy, and unpleasant drug treatment is a significant problem. Many patients stay with therapy for only a few months, and failure to complete it risks not only relapse but also the creation of drug-resistant Mtb. And TB resistant to the first-line drugs isoniazid and rifampicin, called multi-drug-resistant (MDR) TB, has been increasing, particularly in China, India, and the former Soviet Union countries.

While difficult and expensive to treat, MDR-TB can be combated—albeit sometimes less than 60% of the time—by taking one or more of a group of second-line drugs, some with serious side effects, for up to two years. Much more lethal because of the limited treatment options is extensively drug-resistant (XDR) TB. This form is also resistant to second-line fluoroquinoline drugs and one of three injectables—amikacin, capreomycin, or kanamycin.

Thus, the TB problem requires urgent attention. Short course anti-TB regiments initially using at least three first-line drugs (including isoniazid, rifampicin and pyrazinamide) are often not effective due to an increase in the number of tuberculosis strains that have become resistant to current drugs. For example the World Health Organization (WHO) recently reported that the death rate of patients with multi-drug resistant (MDR) tuberculosis in the US was approximately 70%. Current treatment is also very expensive: a 3 drug regimen is needed (more than $500/month cost per patient). Thus the major problems faced in tuberculosis control are poor infrastructures for diagnosis and drug supply. The failure of patients to complete therapy as well as inappropriate monotherapy has led to the emergence and distribution of strains of Mycobacterium tuberculosis resistant to every available chemotherapy (Bloom B R and Murray C J L, Science (1992) 257, 1055-1064). Such organisms will not remain confined to the Third World or to the poor and indigent of developed countries. The recent documentation of the spread of a single clone of multi-drug-resistant Mycobacterium tuberculosis (the “W” strain) throughout the continental United States and Europe highlights the danger of an airborne pathogen in our global society (Bifani P J, et al., JAMA (1996) 275, 452-457).

New therapeutic options are clearly needed to address multiple shortcomings of current therapeutic regimens for TB. One approach to improve the treatment of TB is to improve the efficacy of established TB drugs against susceptible and/or drug-resistant disease.

### SUMMARY OF THE INVENTION

It has now been found that compositions of Timcodar and certain antibiotics are surprisingly effective towards the treatment of mycobacterium infections such as tuberculosis.

### BRIEF DESCRIPTION OF THE DRAWINGS

FIG. 1 depicts A) a scatter plot of Log CFU recovered from the lungs of infected mice either left untreated (Late control) or B) treated with rifampicin (RIF) alone or in combination with Timcodar (TIM).

FIG. 2 depicts the in vitro killing curves of rifampicin (RIF) and RIF+Timcodar (TIM) from a Mtb H37Ra-THP-1 macrophage infection screening assay.

FIG. 3 depicts mean (±S.D.) Timcodr (TIM) plasma concentration-time profiles in C57BL/6 mice following a single oral dose of TIM at either 10 (closed triangles), 100 (closed squares) or 200 (closed circles) mg/kg.

FIG. 4 depicts a scatter plot of Log CFU recovered from the lungs of infected mice either left untreated (Late control) or treated with rifampicin (RIF) alone or in combination with Timcodar (TIM) versus isoniazid (INH) over 4 weeks.

FIG. 5 depicts a scatter plot of Log CFU recovered from the lungs of infected mice either left untreated (Late control) or treated with rifampicin (RIF) and isoniazid (INH) or RIF and INH in combination with Timcodar (TIM) over 9 and 12 weeks.

### DETAILED DESCRIPTION OF THE INVENTION

Applicants have previously described a series of compounds and pharmaceutical compositions, which have been particularly well-suited for treatment of multi-drug resistant cells, for prevention of the development of multi-drug resistance and for use in multi-drug resistant cancer therapy (U.S. Pat. Nos. 5,330,993, 5,620,971, 5,744,485, 5,543,423 and 5,726,184, the disclosures of which are incorporated herein by reference; United States Patent Application No. US20050090482; and PCT Publications: WO92/19593, WO94/07858, WO92/002278, WO95/26337, WO96/15101, and WO94/07858, the disclosures of which are incorporated herein by reference). Currently disclosed are pharmaceutical compositions comprising Timcodar and antibiotics that are particularly effective for the treatment of mycobacterium infections such as TB.

In one aspect, the invention relates to a composition comprising N-benzyl-3-(4-chlorophenyl)-2-[methyl-[2-oxo-2-(3,4,5-trimethoxyphenyl)acetyl]amino]-N-[3-(4-pyridyl)-1-[2-(4-pyridyl)ethyl]propyl]propanamide and a rifamycin antibiotic. In another embodiment, the rifamycin antibiotic is selected from the group consisting of rifampicin, rifabutin, rifalazil, and rifapentine. In another embodiment, the rifamycin antibiotic is rifampicin. In another embodiment, the composition further comprises INH.

In one aspect, the invention relates to a composition comprising N-benzyl-3-(4-chlorophenyl)-2-[methyl-[2-oxo-2-(3,4,5-trimethoxyphenyl)acetyl]amino]-N-[3-(4-pyridyl)-1-[2-(4-pyridyl)ethyl]propyl]propanamide and a diarylquinoline antibiotic. In another embodiment, the diarylquinoline antibiotic is TMC-207. In another embodiment, the composition further comprises INH.

In another aspect, the invention relates to a composition comprising N-benzyl-3-(4-chlorophenyl)-2-[methyl-[2-oxo-2-(3,4,5-trimethoxyphenyl)acetyl]amino]-N-[3-(4-pyridyl)-1-[2-(4-pyridyl)ethyl]propyl]propanamide, a rifamycin antibiotic, and a diarylquinoline antibiotic. In another embodiment, the rifamycin antibiotic is rifampicin. In another embodiment, the diarylquinoline antibiotic is TMC-207. In another embodiment, the rifamycin antibiotic is rifampicin and the diarylquinoline antibiotic is TMC-207. In another embodiment, the composition further comprises INH.

In another aspect, the invention relates to a composition comprising N-benzyl-3-(4-chlorophenyl)-2-[methyl-[2-oxo-2-(3,4,5-trimethoxyphenyl)acetyl]amino]-N-[3-(4-pyridyl)-1-[2-(4-pyridyl)ethyl]propyl]propanamide and INH.

In another aspect, the invention relates to a pharmaceutical composition comprising the composition of any of the ones described above and a pharmaceutical carrier.

In another aspect, the invention relates to a method of treating a subject with a mycobacterium infection comprising administering to the subject an effective amount of the above described pharmaceutical composition. In one embodiment, the mycobacterium infection is tuberculosis.

In another aspect, the invention relates to a method of inhibiting bacterial efflux of a diarylquinoline antibiotic comprising contacting the bacteria with a composition comprising N-benzyl-3-(4-chlorophenyl)-2-[methyl-[2-oxo-2-(3,4,5-trimethoxyphenyl)acetyl]amino]-N-[3-(4-pyridyl)-1-[2-(4-pyridyl)ethyl]propyl]propanamide and a diarylquinoline antibiotic. In one embodiment, the bacteria is Mycobacterium tuberculosis. In another embodiment, the diarylquinoline is TMC-207.

In another aspect, the invention relates to a method of increasing the activity of a rifamycin antibiotic towards mycobacteria comprising contacting the mycobacteria with a rifamycin antibiotic and N-benzyl-3-(4-chlorophenyl)-2-[methyl-[2-oxo-2-(3,4,5-trimethoxyphenyl)acetyl]amino]-N-[3-(4-pyridyl)-1-[2-(4-pyridyl)ethyl]propyl]propanamide. In one embodiment, the rifamycin antibiotic is selected from the group consisting of rifampicin, rifabutin, rifalazil, and rifapentine. In another embodiment, the rifamycin antibiotic is rifampicin. In another embodiment, the mycobacteria is Mycobacterium tuberculosis.

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise stated, all tautomeric forms of the compounds of the invention are within the scope of the invention. For example, Timcodar contains a carbonyl group which can exist in tautomeric forms as shown below:

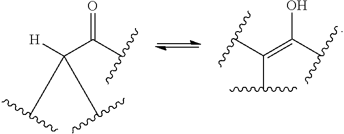

Thus, included within the scope of the invention are tautomers of Timcodar and antibiotics.

Additionally, unless otherwise stated, structures depicted herein are also meant to include compounds that differ only in the presence of one or more isotopically enriched atoms. For example, compounds of formula I, wherein one or more hydrogen atoms are replaced deuterium or tritium, or one or more carbon atoms are replaced by a ¹³C- or ¹⁴C-enriched carbon are within the scope of this invention. Such compounds are useful, for example, as analytical tools, probes in biological assays, or sodium channel blockers with improved therapeutic profile.

As used herein, “Timcodar” or “TIM” is N-benzyl-3-(4-chlorophenyl)-2-[methyl-[2-oxo-2-(3,4,5-trimethoxyphenyl)acetyl]amino]-N-[3-(4-pyridyl)-1-[2-(4-pyridyl)ethyl]propyl]propanamide depicted below

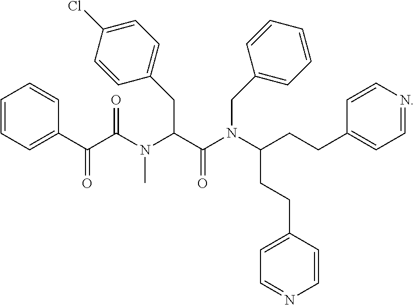

Also included in the term Timcodar or TIM are pharmaceutically acceptable salts of Timcodar, including, for example, a dimesilate salt of Timcodar. Other pharmaceutically acceptable salts are described in the “Pharmaceutically acceptable compositions” section below.

As used herein, the term “rifamycin antibiotic” refers to a group of antibiotics which are synthesized either naturally by the bacterium Amycolatopsis mediterranei, or artificially. They are a subclass of the larger family, Ansamycin. Rifamycins are particularly effective against mycobacteria, and are therefore used to treat tuberculosis, leprosy, and mycobacterium avium complex (MAC) infections. The rifamycin group includes the “classic” rifamycin drugs as well as the rifamycin derivatives rifampicin (or rifampin) (“RIF”), rifabutin, rifalazil, and rifapentine, and pharmaceutically acceptable salts thereof.

As used herein, the term “diarylquinoline antibiotic” refers to a class of antibiotics comprising a quinoline containing two aryl groups, and pharmaceutically acceptable salts thereof.

As used herein, the term “TMC-207” refers to a diarylquinoline antibiotic with anti-tuberculosis properties having the following structure

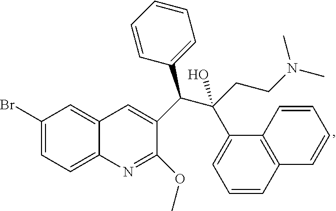

and pharmaceutically acceptable salts thereof.

As used herein, the term “U-100480” refers to an oxazolidinone antibiotic which exhibits in vitro activity against mycobacteria. See Barbachyn, M. R. et al. J. Med. Chem., 1996, 39(3), 680-685, incorporated herein by reference.

As used herein, the term “isoniazid” or “INH” refers to a pyridine antibiotic with anti-tuberculosis properties having the following structure

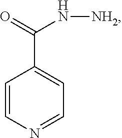

and pharmaceutically acceptable salts thereof.

As used herein, the term “effective amount” of the pharmaceutically acceptable composition is that amount effective for treating or lessening the severity of one or more of mycobacterium infections such as TB.

As used herein, the term “patient” or “subject” means an animal, preferably a mammal, and most preferably a human.

Uses, Formulation and Administration

Pharmaceutically Acceptable Compositions

As discussed above, the present invention provides compositions comprising Timcodar and antibiotics useful for the treatment of diseases caused by mycobacterium infections such as TB. Accordingly, pharmaceutically acceptable compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a subject in need is capable of providing, directly or indirectly, a compound as otherwise described herein, or a metabolite or residue thereof.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or an inhibitorily active metabolite or residue thereof. As used herein, the term “inhibitorily active metabolite or residue thereof” means that a metabolite or residue thereof is also an inhibitor of a voltage-gated sodium ion channel.

Pharmaceutically acceptable salts are well known in the art. For example, S. M. Berge, et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Uses of Compounds and Pharmaceutically Acceptable Compositions

In yet another aspect, a method for the treatment or lessening the severity of a mycobacterium infection such as TB is provided comprising administering an effective amount of a compound, or a pharmaceutically acceptable composition comprising a compound to a subject in need thereof.

The compositions, according to the method of the present invention, may be administered using any amount and any route of administration effective for treating or lessening the severity of a mycobacterium infection such as TB. The exact amount required will vary from subject to subject, depending on the species, age, and general condition of the subject, the severity of the infection, the particular agent, its mode of administration, and the like. The compositions of the invention are preferably formulated in dosage unit form for ease of administration and uniformity of dosage. The expression “dosage unit form” as used herein refers to a physically discrete unit of agent appropriate for the subject to be treated. It will be understood, however, that the total daily usage of the compounds and compositions of the present invention will be decided by the attending physician within the scope of sound medical judgment. The specific effective dose level for any particular subject or organism will depend upon a variety of factors including the disorder being treated and the severity of the disorder; the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed, and like factors well known in the medical arts.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a composition of the present invention, it is often desirable to slow the absorption of the composition from subcutaneous or intramuscular injection. This may be accomplished by the use of a liquid suspension of crystalline or amorphous material with poor water solubility. The rate of absorption of the composition then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

It will also be appreciated that pharmaceutically acceptable compositions of the present invention can be employed in combination therapies, that is, the pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated”. For example, exemplary additional therapeutic agents include, but are not limited to: nonopioid analgesics (indoles such as Etodolac, Indomethacin, Sulindac, Tolmetin; naphthylalkanones such as Nabumetone; oxicams such as Piroxicam; para-aminophenol derivatives, such as Acetaminophen; propionic acids such as Fenoprofen, Flurbiprofen, Ibuprofen, Ketoprofen, Naproxen, Naproxen sodium, Oxaprozin; salicylates such as Asprin, Choline magnesium trisalicylate, Diflunisal; fenamates such as meclofenamic acid, Mefenamic acid; and pyrazoles such as Phenylbutazone); or opioid (narcotic) agonists (such as Codeine, Fentanyl, Hydromorphone, Levorphanol, Meperidine, Methadone, Morphine, Oxycodone, Oxymorphone, Propoxyphene, Buprenorphine, Butorphanol, Dezocine, Nalbuphine, and Pentazocine). Additionally, nondrug analgesic approaches may be utilized in conjunction with administration of one or more compounds of the invention. For example, anesthesiologic (intraspinal infusion, neural blocade), neurosurgical (neurolysis of CNS pathways), neurostimulatory (transcutaneous electrical nerve stimulation, dorsal column stimulation), physiatric (physical therapy, orthotic devices, diathermy), or psychologic (cognitive methods-hypnosis, biofeedback, or behavioral methods) approaches may also be utilized. Additional appropriate therapeutic agents or approaches are described generally in The Merck Manual, Seventeenth Edition, Ed. Mark H. Beers and Robert Berkow, Merck Research Laboratories, 1999, and the Food and Drug Administration website, www.fda.gov, the entire contents of which are hereby incorporated by reference.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention or pharmaceutically acceptable compositions thereof may also be incorporated into compositions for coating an implantable medical device, such as prostheses, artificial valves, vascular grafts, stents and catheters. Accordingly, the present invention, in another aspect, includes a composition for coating an implantable device comprising a compound of the present invention as described generally above, and in classes and subclasses herein, and a carrier suitable for coating said implantable device. In still another aspect, the present invention includes an implantable device coated with a composition comprising a compound of the present invention as described generally above, and in classes and subclasses herein, and a carrier suitable for coating said implantable device. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

### EXAMPLES

### Materials and Methods

Compounds: Timcodar was prepared at Vertex Pharmaceuticals as described in U.S. Pat. Nos. 5,330,993 and 5,620971 and PCT Publications WO92/19593, WO94/07858 and WO92/00278; the disclosures of which are incorporated herein by reference. Isoniazid (INH), ethionamide, rifampicin (RIF), acriflavin, and ethidium bromide were purchased from Sigma Chemical Co. (St. Louis, Mo.). Gatifloxacin was provided by Bristol Myers Squibb. Moxifloxacin and rifapentine were from Sequoia Research Products (Pangbourne, UK). Linezolid and U-100480 were provided by Upjohn Pharmacia and levofloxacin was provided by RW Johnson Pharmaceutical Research Institute. TMC-207 was provided by Shanghai Chem. Partner.

Susceptibility testing: All drugs were dissolved in 100% dimethyl sulfoxide (DMSO) and diluted in 7H10 broth with 10% OADC and 0.05% Tween 80. Drugs, prepared at 4× the maximal concentration in either 7H10 alone, or in 7H10+10 μg/ml of TIM, were added to the first well and serially diluted two-fold in polystyrene 96-well round bottom plates (Corning Inc., Corning, N.Y.). Mtb ATCC 35801 (strain Erdman), frozen at 5×10⁷ CFU/ml, was diluted to a final concentration of 1.25×10⁵ CFU/ml in 7H10 broth and 50 μl was added to each drug dilution well. The actual inoculum was determined by titration and plating on 7H10 agar. Plates were covered with SealPlate adhesive sealing film (Excel Scientific, Wrightwood, Calif.) and incubated at 37° C. in ambient air for 18 days prior to reading. The MIC was defined as the lowest concentration of agents yielding no visible turbidity. Each drug was tested in duplicate.

Mtb H37Ra—THP-1 Macrophage Infection Screening Assay Protocol

Materials:
1) RPMI 1640 with L-glutamine and HEPES (Lonza BioWhittaker, #12-115F) medium #1.
2) RPMI 1640 without phenol red, L-glutamine or HEPES (Lonza BioWhittaker, #12-918F) medium #2.
3) Fetal Bovine Serum, defined, (Hyclone, #SH30070.03).
4) 200 mM L-glutamine (Cellgro, #25-005-C1).
5) β-mercaptoethanol (Sigma, M-7522).
6) 1 M HEPES (Cellgro, #25-060-C1).
7) PMA (Sigma, P8139) 5000× stock in DMSO (Sigma, D2650).
8) F-150 vent cap Flasks.
9) Hemocytometer counting chamber (Hausser #3500).
10) 96-well, white sided, clear bottom, sterile microtiter culture plates (Costar #3903).
11) 7H9 broth (BD271310) with 0.2% glycerol, 0.05% Tween 80, 10% ADC (BD #X) and 20 ug/ml kanamycin.
12) 7H11 agar (BD283810) plates with 0.2% glycerol, 10% OADC (BD BBL 212351), 40 ug/ml kanamycin, 50 ug/ml cyclohexamide (Sigma, C1988) and 20 ug/ml amphotericin B (Fungizone, Gibco Cat#15290-018).
13) Filter sterilized Krebs/tween80 Buffer (120 mM NaCl; 4.9 mM KCl; 1.2 mM MgSO₄; 1.7 mM KH₂PO₄; 8.3 mM Na₂HPO₄; 10 mM Glucose; 0.02% Tween 80; 0.5% BSA).
14) Sterile disposable 10 μl inoculation loops.
15) Sterile microfuge tubes with o-ring seal.
16) Bacterial cell counting chamber (Hausser #3900).
17) Bath Sonicator.

Maintenance and Expansion of THP-1 cells: THP-1 stocks are maintained in HEPES-buffered (25 mM) RPMI-1640 media (with phenol red and 2 mM L-glutamine; medium #1) supplemented with 10% FBS and 0.05 mM β-mercaptoethanol. It is essential to maintain the culture density between 2×10⁵ and 8×10⁵ cells/ml. Do not let cultures exceed 1×10⁶ cells per ml. Typically the cells are split to a density of 2.0×10⁵ twice per week.

Culture of Mtb H37Ra 4917: Cultures are maintained in 7H9 broth supplemented with 0.2% glycerol, 0.05% Tween 80, 10% ADC, and 20 ug/ml kanamycin, (25 ml in a filter-cap 125 ml plastic Erlenmeyer flask), 37° C., static incubation. These cultures are passed weekly by transferring a 20% volume to a fresh volume of broth (initial OD₆₂₀ ˜0.04; at 7 days OD₆₂₀ ˜0.20-0.25). Five days prior to infection 0.5-1 ml of a mid-log phase culture (OD₆₂₀ ˜0.20) is spread onto 7H11 agar supplemented with 0.2% glycerol+10% OADC, 40 ug/ml kanamycin, 50 ug/ml cyclohexamide and 20 ug/ml amphotericin B, and incubated at 37° C.

Infection Assay

Day (−1)

One day prior to use of the cells, feed the THP-1 cells with ˜30% volume of complete RPMI media, adjusting the cell density to 2−3×10⁵ cells/ml.

Day 1

Plating and Differentiation of THP-1 Monocytes
1) Pool and count cells using a hemocytometer. Harvest cells by centrifugation at 200×g for 10 min. Re-suspend the cells to a density of 4×10⁵ cells/ml in HEPES (10 mM)-buffered RPMI-1640 media (without phenol red; medium #2) supplemented with 10% FBS, 2 mM L-glutamine, 0.05 mM β-mercaptoethanol and 100 nM PMA (diluted from 5000× stock).
2) Dispense 100 μL of the cell suspension into each well of the required number of 96-well plates (Costar 3903).
3) Differentiate for 60 to 72 hours (typically plated on a Friday for use on Monday).

Day 4

Preparation of Mtb H37Ra Cell-Suspension
1) Scrape a loopful of bacterial cells from a plate and collect in sterile microfuge tube with o-ring seal containing 1 ml Krebs+0.02% Tween-80.
2) Sonicate solution for 10 seconds (in water bath sonicator); rest for 15 seconds and repeat 3 times. The solution is allowed to rest at room temp for 1 hour to settle larger clumps. 0.5 ml is carefully removed from the top and added to an additional 0.5 ml Krebs+Tween-80 (total volume is again 1 ml).
3) Dilute a sample 1:10 in Krebs+0.02% Tween-80. Sonicate for 10 seconds and count immediately in bacterial counting chamber.

Infection
1) Immediately prior to diluting M tb, sonicate the bacterial suspension for 10 seconds.
2) Dilute bacterial cells to a density of 4×10⁵ cells/ml for an MOI of 1:1 into 25 mM HEPES-buffered RPMI-1640 media (without phenol red; medium #2) supplemented with 10% FBS, L-glutamine, 0.05 mM β-mercaptoethanol. (if additional MOIs are used prepare bacterial suspensions scaled to the infection rate; for example 8×10⁵ cells/ml for an MOI of 2:1, 2×10⁶ cells/ml for an MOI of 5:1).
3) Remove the media above THP-1 cells with a multi-channel pipettor and replace with 100 μl of the bacterial suspension. Incubate 2 h at 37° C.
4) Test compounds are dispensed into sterile round bottom 96 well plates at the desired concentration in 1 μl volumes of DMSO (0.5% DMSO final). Just prior to treatment of cells with the compound, dispense 250 μl of sterile media into wells containing test compounds and mix. Compounds should be arranged in a template identical to the intended plate maps of the infection assay wells so that the medium + compound from an entire plate can be directly transferred to the test plates.
5) Remove supernatant containing uningested Mtb from each well and replace with 100 μl fresh media. Remove supernatant a second time from each well and replace with 50 μl fresh media and 50 μl of media containing test compounds (from step 4). Total dilution for the test compound is 500×.
6) Measure baseline luciferase in a whole plate containing 48 wells of uninfected THP-1 cells and 48 wells of freshly infected THP-1 cells each treated with DMSO. Add 100 ul of Bright Glow reagent to each well, incubate for 10 minutes at RT, cover with adhesive top seal and read luminescence in Tecan Spectrafluor plus, at a gain of 150 at maximum integration time.
7) Incubate remaining plates at 37° C., 5% CO₂, in humidified chamber.
8) Measure endpoint luciferase of all test and control plates 5 days (120 hours) after infection. Add 100 ul of Bright Glow reagent to each well, incubate for 10 minutes at RT, cover with adhesive top seal and read luminescence in Tecan Spectrafluor+, at a gain of 150 at maximum integration time. Stagger timing of addition and reading by 3 minutes between plates.

Mouse Infection Model

Mice. Six-week old female C57BL/6 mice were purchased from Jackson Laboratories, Bar Harbor, Me. and were maintained within the ABSL-3 at the Syracuse Va. Medical Center's Veterinary Medical Unit, Syracuse, N.Y. All animal procedures were approved by the Subcommittee for Animal Studies (SAS). Mice were housed in micro-isolator cages (lab products inc, Maywood, N.J.) and maintained with water and Prolab RMH 3000 rodent chow (Purina, St. Louis, Mo.).

Isolate. Mtb ATCC 35801 (strain Erdman) was obtained from the American Type Culture Collection (ATCC) Manassas, Va. and grown in modified 7H10 broth (pH 6.6; 7H10 agar formulation with agar and malachite green omitted) with 10% OADC (oleic acid, albumin, dextrose, catalase) enrichment (BBL Microbiology Systems, Cockeysville, Md.) and 0.05% Tween 80 for 5-10 days on a rotary shaker at 37° C. The culture was diluted to 100 Klett units (equivalent to 5×10⁷ colony forming units (CFU)) per ml (Photoelectric Colorimeter; Manostat Corp., New York, N.Y.). The culture was frozen at −70° C. until use. On the day of infection the culture was thawed and sonicated. The final inoculum size was determined by titration, in triplicate, on 7H10 agar plates (BBL Microbiology Systems, Cockeysville, Md.) supplemented with 10% OADC enrichment. The plates were incubated at 37° C. in ambient air for 4 weeks.

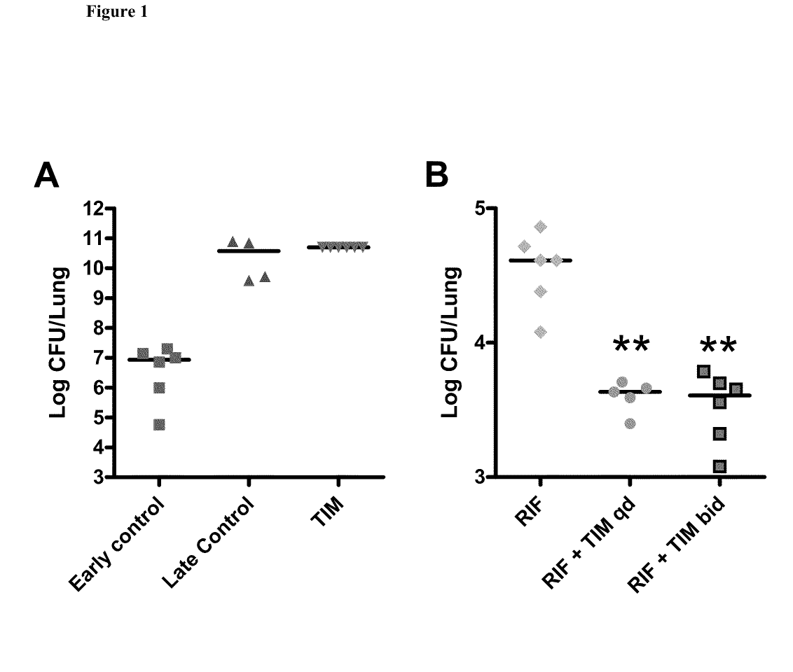

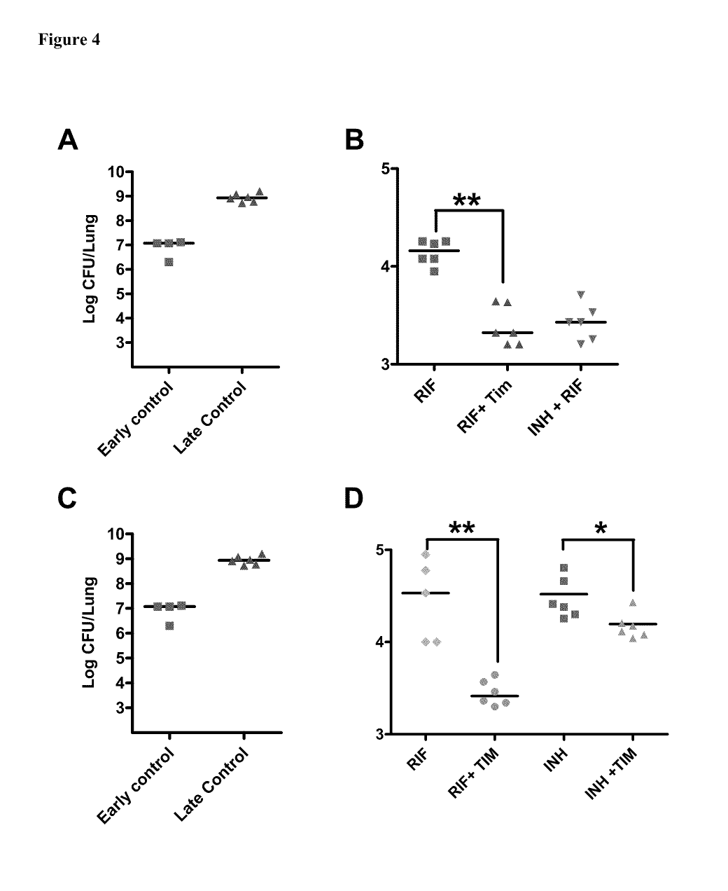

Infection study. Six-week old female C57BL/6 mice were infected intranasally with approximately 10⁶ CFU of Mtb Erdman. Mice were randomly assigned to groups of 6 mice as indicated in the figure legends. Treatment was started 7 days post-infection and was continued for 4 weeks (5 days on and 2 days off treatment) or 9 and 12 weeks (5 days on and 2 days off treatment). TIM and RIF were dissolved in 0.5% carboxymethylcellulose for the first and second study (FIGS. 1 and 4A & B) and in the third, RIF was dissolved in DMSO and diluted in distilled water (final concentration of DMSO was 4%; FIGS. 4C and D). INH was dissolved in 0.5% carboxymethylcellulose for the second study (FIGS. 4A and B) and distilled water in the third (FIGS. 4C and D). TIM, RIF and INH were dosed at 200, 10 and 25 mg/kg, respectively. Note that RIF and INH efficacy appeared to be unaffected by vehicle. TIM was given in the morning and RIF or INH were administered 5-6 hours post-TIM treatment. An Early Control (EC) group was euthanized at the initiation of therapy to determine the infection load. A Late Control (LC) group was utilized to confirm virulence; LC mice were moribund and needed to be euthanized at 14 days post-infection.

Statistical evaluation. To compare the viable cell counts recovered from the right lungs of mice, the numbers were first converted into log cfu (log₁₀). Due to the small sample size and the consequent need to protect against deviations from normality, the Mann-Whitney test was performed to determine statistical differences between control and treatment groups.

Pharmacokinetic Studies

To determine the oral exposure of TIM in C57BL/6 mice; 10, 100 or 200 mg/kg was administered by oral gavage to mice (10 mL/kg) in an aqueous solution of 0.5% carboxymethylcellulose. Whole blood was sampled by retro-orbital bleeding (three mice per timepoint) at 0.25, 0.5, 1, 1.5, 2, 3, 4, 6, 8 and 24 hours after dosing and plasma was obtained following centrifugation of blood samples at 3000×g for 2 minutes. Samples were extracted using acetonitrile (4:1) containing an analytical internal standard (IS), vortexed for 5 minutes, centrifuged at 3,000×g for 20 minutes and supernatants were transferred to 96-well plates for quantitative LC/MS. Samples and standards (which were prepared in plasma matrix and extracted as described above), were analyzed using a Shimadzu UFLC system with a Sepax HP C18 column followed by MS analysis using a SCIEX API 4000 (Applied Biosystems) tandem triple quadrupole mass spectrometer in ESI Ionization Mode and MRM Scan Mode. Quantitation of samples and standards were determined relative to the IS. Pharmacokinetic parameters were determined by non-compartmental analysis of the plasma concentration data using WinNonlin software (Pharsight Corp., Mountain View, Calif.). In the pharmacokinetic studies where TIM was co-administered with RIF, TIM (200 mg/kg) was administered orally to mice six hours prior to the oral administration of RIF (10 mg/kg) dissolved in an aqueous solution of 0.5% carboxymethylcellulose. Blood sampling, plasma extraction and quantitation of RIF by LC/MS analysis and pharmacokinetic analysis of the data were carried out as described above.

### Results and Discussion

TIM at 10 μg/ml, a level achievable in mice by orally dosing did not affect the antimicrobial activities of a diverse set of anti-mycobacterial drugs or the dye acriflavin (Table 1).

TABLE 1
In vitro activity of Timcodar in combination with antibiotics
against Mycobacterium tuberculosis Erdman strain.

MIC* (μg/ml)

| Compound | No TIM | +TIM 10 μg/ml |
|---|---|---|
| Isoniazid | 0.06, 0.03 | 0.03, 0.03 |
| gatifloxacin | 0.03, 0.015 | 0.015, 0.015 |
| moxifloxacin | 0.03, 0.03 | 0.03, 0.03 |
| levofloxacin | 0.13, 0.13 | 0.13, 0.13 |
| ethionamide | 32, 32 | 32, 32 |
| rifampicin | 0.004, 0.002 | 0.002, 0.002 |
| Rifapentine | 0.002, 0.002 | 0.002, 0.002 |
| Linezolid | 0.5, 1 | 0.25, 0.5 |
| Acriflavin | 1, 1 | 0.5, 0.5 |
| U-100480 | 0.25, 0.25 | 0.25, 0.25 |
| ethidium bromide | 4, 4 | 1, 1 |
| TMC-207 | 0.06, 0.06 | 0.004, 0.004 |

*replicate values shown

As can be seen from Table 1, TIM had no in vitro effect on the activity of isoniazid, gatifloxacin, moxifloxacin, levofloxacin, ethionamid, rifampicin, rifapentine, linezolid, acriflavin, and U-100480 against Mtb. A four-fold potentiating effect was observed with the intercalator ethidium bromide, a known promiscuous efflux pump substrate, suggesting that TIM has some effect on Mtb that is consistent with efflux inhibition. Surprisingly, a 15-fold potentiating effect on the activity of TMC-207 against Mtb was observed. This makes the combination of TIM and TMC-207 especially effective for the treatment of patients with TB.

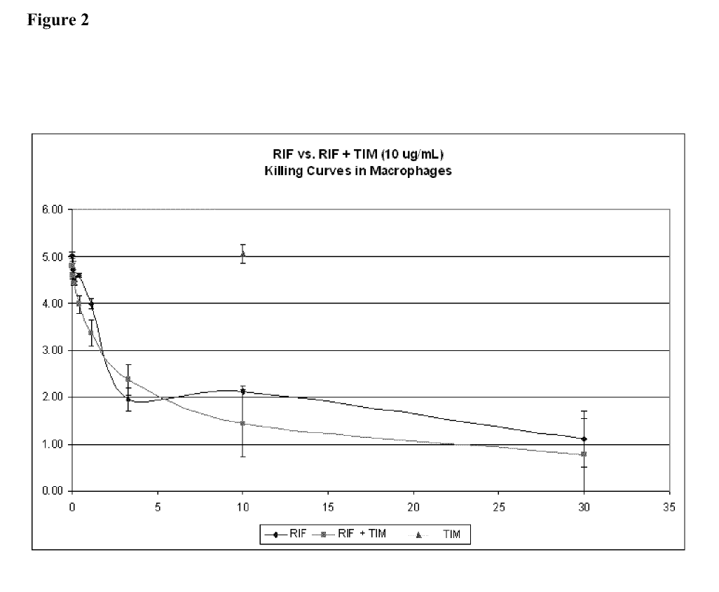

In a macrophage assay, TIM had no significant in vitro effect on the inhibitory activity of RIF against Mtb based on colony forming units (cfus), as can be seen by the data in Table 2 and FIG. 2.

TABLE 2
In vitro activity of Timcodar in combination with RIF against
Mycobacterium tuberculosis Erdman strain in macrophages.

| Compound | Drug conc. (ug/mL) | Log10 CFU | SEM |
|---|---|---|---|
| RIF | 30 | 1.10 | 0.59 |
| RIF | 10 | 2.12 | 0.12 |
| RIF | 3.3 | 1.94 | 0.24 |
| RIF | 1.1 | 3.98 | 0.12 |
| RIF | 0.4 | 4.58 | 0.04 |
| RIF | 0.1 | 4.47 | 0.10 |
| RIF | 0.04 | 4.71 | 0.17 |
| RIF | 0 | 5.02 | 0.06 |
| RIF + TIM | 30 | 0.77 | 0.77 |
| RIF + TIM | 10 | 1.43 | 0.72 |
| RIF + TIM | 3.3 | 2.37 | 0.32 |
| RIF + TIM | 1.1 | 3.36 | 0.28 |
| RIF + TIM | 0.4 | 3.98 | 0.20 |
| RIF + TIM | 0.1 | 4.43 | 0.02 |
| RIF + TIM | 0.04 | 4.59 | 0.08 |
| RIF + TIM | 0 | 4.79 | 0.05 |
| TIM | 10 | 5.04 | 0.20 |

Even though TIM alone had no significant effect on the in vitro growth of Mtb (Figurel), surprisingly, TIM in combination with RIF, potentiated the in vivo antibacterial activity of RIF by one log CFU (FIG. 1) in mice. One would not have expected this pronounced in vivo effect since TIM had no significant in vitro effect on the activity of RIF against Mtb (Tables 1 and 2). Indeed, TIM in combination with RIF, is as potent as the combination of RIF and INH (FIG. 4A). While TIM does enhance INH activity in vivo it does not do so to the same extent as RIF (FIG. 4B). However, the effect is still statistically significant and it is envisioned by the inventors that there may be synergy in a TIM/RIF/INH combination that could be clinically useful, perhaps even shortening therapy or addressing resistance to either of those drugs.

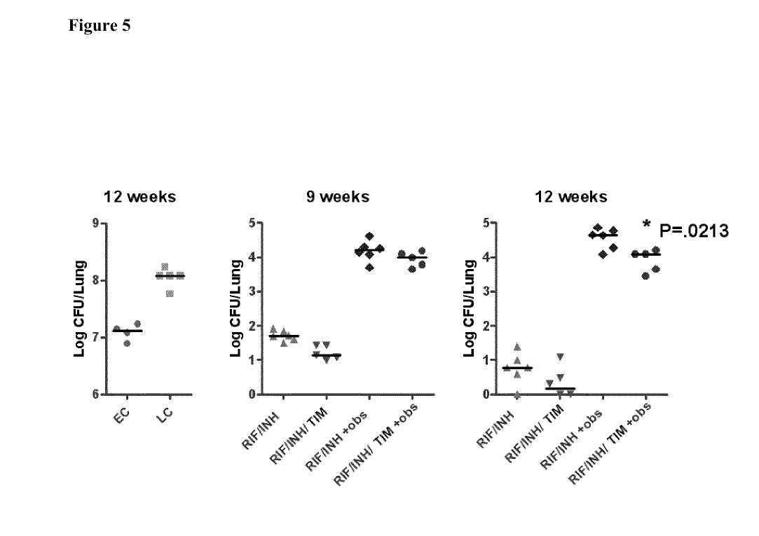

When the in vivo mouse experiment was run for 9 and 12 weeks, results showed that TIM had a statistically significant (as determined by a P value of less than 0.05) potentiating effect on a RIF and INH combination (FIG. 5).

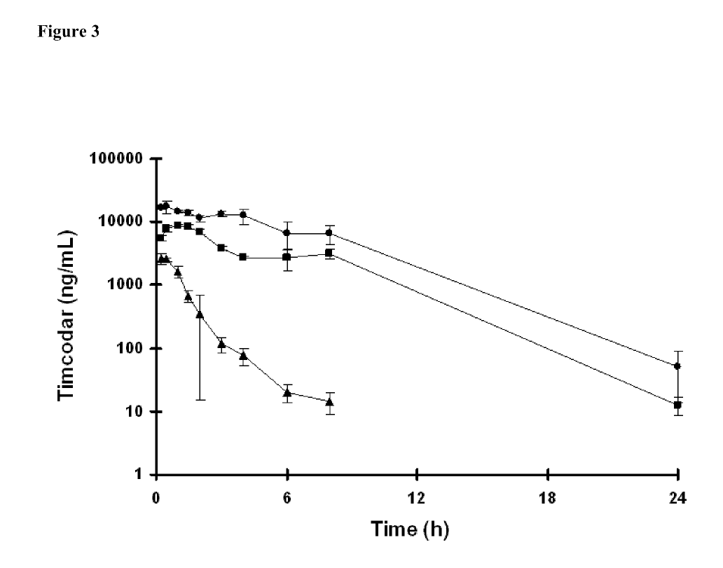

For the in vivo evaluations, TIM was maintained at 5-15 μg/ml for over 16 hours, similar to the concentration used to evaluate TIM in vitro (FIG. 3). Interestingly, pharmacokinetic parameters of RIF in plasma were unaffected by the co-dosing of TIM (Table 3), suggesting that TIM may be acting more specifically as an efflux inhibitor at the level of infected tissue, potentiating the activity of RIF.

TABLE 3
Pharmacokinetic parameters of RIF alone, and in combination
with TIM, in the plasma of C57BL/6 mice.

| | RIF | | | TIM | | |
|---|---|---|---|---|---|---|
| | AUC_{inf} | Cₘₐₓ | | AUC_{inf} | Cₘₐₓ | |
| | Plasma | Plasma | T_{1/2} | Plasma | Plasma | T_{1/2} |
| Drug# | (μg · h/ml) | (μg/ml) | (h) | (μg · h/ml) | (μg/ml) | (h) |
| RIF, 10 mg/kg | 152.0 | 19.8 | 4.9 | | | |
| RIF, 10 | 153.0 | 14.9 | 6.1 | 84.4 | 10.4 | 3.6 |
| mg/kg + | | | | | | |
| TIM*, 200 | | | | | | |
| mg/kg | | | | | | |
| TIM, 10 mg/kg | | | | 3.4 | 2.7 | 1.6 |
| TIM, 100 | | | | 60.4 | 8.9 | 2.4 |
| mg/kg | | | | | | |
| TIM 200 | | | | 137 | 14.7 | 2.4 |
| mg/kg | | | | | | |

*TIM dosed 6 hours prior to RIF
#compounds dosed in 0.5% methyl cellulose in water

Although not wanting to be bound by theory, it is believed that potential mechanisms for TIM potentiation of anti-tuberculosis activity of antibiotics include:

a) promoting uptake into Mtb during infection;

b) increasing oral absorption and systemic distribution of antibiotics in infected tissues; and/or

c) modulating metabolism of other drugs by drug-drug interactions.

It is expected that the potentiating mechanism(s) of TIM will likely be different for different antibiotic drugs.

Because of the potentiating effect TIM has on RIF, TMC-207, and INH, it is envisioned by the inventors that in one embodiment, a composition comprising any combination of TIM with RIF, TMC-207, and INH would be very effective for the treatment of TB.

Many modifications and variations of the embodiments described herein may be made without departing from the scope, as is apparent to those skilled in the art. The specific embodiments described herein are offered by way of example only.

We claim:

1. A composition comprising N-benzyl-3-(4-chlorophenyl)-2-[methyl-[2-oxo-2-(3,4,5-trimethoxyphenyl)acetyl]amino]-N-[3-(4-pyridyl)-1-[2-(4-pyridyl)ethyl]propyl]propanamide and a rifamycin antibiotic.

2. The composition of claim 1, wherein the rifamycin antibiotic is selected from the group consisting of rifampicin, rifabutin, rifalazil, and rifapentine.

3. The composition of claim 1, wherein the rifamycin antibiotic is rifampicin.

4. The composition of claim 3, further comprising INH.

5. A composition comprising N-benzyl-3-(4-chlorophenyl)-2-[methyl-[2-oxo-2-(3,4,5-trimethoxyphenyl)acetyl]amino]-N-[3-(4-pyridyl)-1-[2-(4-pyridyl)ethyl]propyl]propanamide and a diarylquinoline antibiotic.

6. The composition of claim 5, wherein the diarylquinoline antibiotic is TMC-207.

7. The composition of claim 6, further comprising INH.

8. A composition comprising N-benzyl-3-(4-chlorophenyl)-2-[methyl-[2-oxo-2-(3,4,5-trimethoxyphenyl)acetyl]amino]-N-[3-(4-pyridyl)-1-[2-(4-pyridyl)ethyl]propyl]propanamide, a rifamycin antibiotic, and a diarylquinoline antibiotic.

9. The composition of claim 8, wherein the rifamycin antibiotic is rifampicin.

10. The composition of claim 8, wherein the diarylquinoline antibiotic is TMC-207.

11. The composition of claim 8, wherein the rifamycin antibiotic is rifampicin and the diarylquinoline antibiotic is TMC-207.

12. The composition of claim 11, further comprising INH.

13. A composition comprising N-benzyl-3-(4-chlorophenyl)-2-[methyl-[2-oxo-2-(3,4,5-trimethoxyphenyl)acetyl]amino]-N-[3-(4-pyridyl)-1-[2-(4-pyridyl)ethyl]propyl]propanamide, a rifamycin antibiotic, and INH.

14. A pharmaceutical composition comprising the composition of any one of claims 1 to 13 and a pharmaceutical carrier.

15. A method of treating a subject with a mycobacterium infection comprising administering to the subject an effective amount of the composition of any one of claims 1 to 14.

16. The method of claim 15, wherein the mycobacterium infection is Mycobacterium tuberculosis.

17. A method of inhibiting mycobacterial efflux of a diarylquinoline antibiotic comprising contacting the mycobacteria with a composition of claim 5.

18. The method of claim 17, wherein the bacteria is Mycobacterium tuberculosis.

19. The method of claim 17, wherein the diarylquinoline antibiotic is TMC-207.

20. A method of increasing the activity of a rifamycin antibiotic towards mycobacteria comprising contacting the mycobacteria with a composition of claim 1.

21. The method of claim 20, wherein the rifamycin antibiotic is selected from the group consisting of rifampicin, rifabutin, rifalazil, and rifapentine.

22. The method of claim 20, wherein the rifamycin antibiotic is rifampicin.

23. The method of claim 20, wherein the mycobacteria is Mycobacterium tuberculosis.
